(19) United States
(12) Patent Application Publication
(10) Pub. No.: US 2022/0139507 A1
(43) Pub. Date: May 5, 2022

(54) USING MEDICAL TEST RESULTS TO TRIGGER ROUTINE FOR MEDICAL SUPPLY REPLENISHMENT

(71) Applicant: Hewlett-Packard Development Company, L.P., Spring, TX (US)

(72) Inventors: Mithra Vankipuram, Palo Alto, CA (US); Eric Faggin, Palo Alto, CA (US); Alexander Thayer, Palo Alto, CA (US); Josep-Maria Serra, San Diego, CA (US)

(73) Assignee: Hewlett-Packard Development Company, L.P., Spring, TX (US)

(21) Appl. No.: 17/432,117

(22) PCT Filed: Jun. 19, 2019
(86) PCT No.: PCT/US2019/037906

§ 371 Date: Aug. 19, 2021

(51) Int. Cl. G16H 10/40; G16H 40/67; G16H 40/20; G16H 50/80

(52) U.S. Cl. CPC G16H 10/40; G16H 50/80; G16H 40/20; G16H 40/67

(57) ABSTRACT

Examples are described herein for using medical test results to trigger routine for medical supply replenishment. In various examples, based on a result of a medical test administered to a subject at a first medical facility, it may be determined that the subject has an exogenous medical condition. In response to determining that the subject has the exogenous medical condition, a message may be transmitted over a computer network from a computing device associated with the first medical facility to a remote computing device associated with a second medical facility. The message may trigger the remote computing device to initiate a routine to replenish medical supplies associated with the exogenous medical condition at the second medical facility.

### BACKGROUND

Outbreaks of exogenous medical conditions such as contagious diseases, waterborne illnesses, or other environmentally-triggered medical conditions can occur at any time without warning. When an unexpected outbreak of a particular medical condition occurs, medical facilities may be caught off guard by the sudden surge in patients or “subjects.”

### BRIEF DESCRIPTION OF THE DRAWINGS

Features of the present disclosure are illustrated by way of example and not limited in the following figure(s), in which like numerals indicate like elements.

FIG. 1 is a drawing of an example environment in which selected aspects of the present disclosure may be implemented.

FIG. 2 depicts an example of a medical testing device configured with selected aspects of the present disclosure.

FIG. 3 schematically demonstrates how detected co-occurrence of an exogenous medical condition within a geographic area may trigger various aspects of the present disclosure.

FIG. 4 depicts an example method of practicing selected aspects of the present disclosure.

FIG. 5 depicts an example method of practicing selected aspects of the present disclosure.

FIG. 6 depicts an example computer architecture.

### DETAILED DESCRIPTION

For simplicity and illustrative purposes, the present disclosure is described by referring mainly to an example thereof. In the following description, numerous specific details are set forth in order to provide a thorough understanding of the present disclosure. It will be readily apparent however, that the present disclosure may be practiced without limitation to these specific details. In other instances, some methods and structures have not been described in detail so as not to unnecessarily obscure the present disclosure.

Additionally, it should be understood that the elements depicted in the accompanying figures may include additional components and that some of the components described in those figures may be removed and/or modified without departing from scopes of the elements disclosed herein. It should also be understood that the elements depicted in the figures may not be drawn to scale and thus, the elements may have different sizes and/or configurations other than as shown in the figures.

Outbreaks of exogenous medical conditions such as contagious diseases, waterborne illnesses, or other environmentally-triggered medical conditions can occur at any time without warning. Without knowing when an outbreak of a particular medical condition will occur, it is difficult for a medical facility to know how big an inventory it needs of medical supplies associated with the particular medical condition. When an unexpected outbreak of a particular medical condition occurs, medical facilities may be caught off guard by the sudden surge in patients or “subjects,” and may find their inventory of medical supplies for testing and/or treating the medical condition to be lacking.

Examples are described herein that relate to automatically investigating and/or acting upon the need for medical supplies based on medical test results. More particularly, various examples described herein are directed to triggering, at one medical facility, investigation and/or replenishment of medical supplies related to a exogenous medical condition based on results of a medical test administered at another medical facility that indicate a subject has the exogenous medical condition.

In some examples, a medical test may be administered with a medical testing device that is designed to test for a specific exogenous medical condition. The medical testing device may transmit a message indicative of a result of the medical test to a computing device, which may be located at the same medical facility or at a remote medical facility, e.g., in the same geographic region. If the medical test result is positive, indicating the subject has the exogenous medical condition, the computing device may trigger, or may cause another computing device (e.g., at a remove medical facility) to trigger, a routine to replenish medical supplies associated with the exogenous medical condition.

These medical supplies may include, for instance, fungible components of medical testing devices that test for the exogenous medical condition. The fungible components may include, for instance, a disposable sample compartment or cartridge that is removably installable in a base component of the medical testing device, or another disposable component of the medical testing device such as a swab, test strip, chemical reagent (e.g., to interact with a sample of a subject), and the like. Medical supplied may additionally or alternatively include supplies for treating the exogenous medical condition and/or its symptoms, such as medications, topical ointments, pain killers, etc.

Techniques described herein do not rely on information about individual patients. Rather, messages exchanged between medical testing devices and/or computing devices of medical facilities may simply signal a potential outbreak of an exogenous medical condition, with the patient's identifying information being left out. These anonymized messages may nonetheless trigger, at other medical facilities, automatic investigation and/or replenishment of medical supplies in order to stay ahead of the outbreak, without the need to know which individuals have the disease.

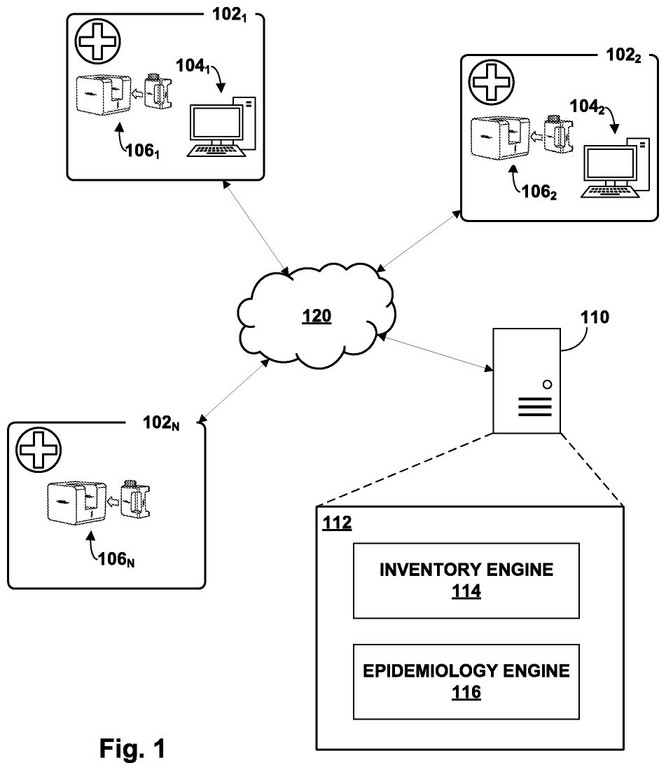

FIG. 1 is a schematic drawing of an example environment in which selected aspects of the present disclosure may be implemented. A plurality of medical facilities 102_{1-N} are distributed throughout a geographic area. A “medical facility” as used herein refers to a location or facility at which individuals (“patients,” “subjects”) can be medically serviced, e.g., by being tested for medical conditions, treated for symptoms and/or medical conditions, etc. A medical facility may include, for instance, a general practitioner's office, a pediatrician's office, a quick-service health clinic of the type often found in grocery stores or drug stores, a hospital, an immediate care office, a more specialized clinic, and so forth. Each medical facility 102 may include an inventory of medical supplies for testing for and/or treating medical conditions.

First medical facility 102₁ and second medical facility 102₂ include, respectively, computing devices 104₁, 104₂. Computing devices 104 may include, for example: a desktop computing device, a laptop computing device, a tablet computing device, a mobile phone computing device, a standalone interactive speaker, a smart appliance such as a smart television (or a standard television equipped with a networked dongle with automated assistant capabilities), and/or a wearable apparatus of the user that includes a computing device (e.g., a watch of the user having a computing device, glasses of the user having a computing device, a virtual or augmented reality computing device). Additional and/or alternative computing devices may be provided. Computing devices 104 and/or other components of different medical facilities 102 are communicatively coupled with each other via wired and/or wireless computing network(s) indicated generally at 120.

Medical facilities 102_{1-N} also include respective medical testing devices 106_{1-N}. In some examples, in a given medical facility 102, medical testing device 106 may be communicatively coupled with computing device 104 using various wired or wireless technologies, such as universal serial bus (“USB”), wireless local area networks (“LAN”) that employ technologies such as the Institute of Electrical and Electronics Engineers (“IEEE”) 802.11 standards, personal area networks, mesh networks, high-definition multimedia interface (“HDMI”), and so forth.

In some examples, when medical testing device 106 is operated to administer a medical test to a patient, a result of the medical test may be transmitted by medical testing device 106 to computing device 104. Computing device 104 may perform various actions based on the medical test result, such as storing data indicative of the result in an electronic health record (“EHR”) associated with the patient. In other examples, medical testing device 106 may provide output (e.g., audible, visual) indicative of the medical test result. An individual such as a nurse, front desk worker, clinician, medical technician, etc., working at the medical facility 102 may manually input this information at a user interface of computing device 104. In some examples, when the result indicates the patient has an exogenous medical condition, computing device 104 may trigger a routine to replenish medical supplies associated with an exogenous medical condition.

In some examples, medical testing device 106 itself may transmit or otherwise take action based on a result of a medical test it administered. For example, in medical facility 102_{N}, medical testing device 106_{N} is a standalone device that may transmit data indicative of a medical test result through network(s) (e.g., 120) to remote computing devices (e.g., 104₁, 104₂) and/or to other standalone medical testing devices.

In some examples, the messages exchanged between medical facilities that indicate occurrence of an exogenous medical condition may be anonymized, e.g., patient information may be scrubbed, before those messages are transmitted. In some examples, a message may be anonymized simply by ensuring that identifying information of a patient that tested positive for the medical condition is not contained in the message.

In some examples, additional or alternative measures may be taken to ensure patient anonymity. For example, a positive test result may not immediately cause a computing device 104 or a medical testing device 106 to transmit a message to other nearby medical facilities. Rather, an artificial delay may be taken before the message is sent, e.g., to reduce the likelihood that the patient's physical presence at the medical facility could be linked to their positive test for the exogenous medical condition. This artificial delay may be a predetermined time period, a randomly-selected time period, or even a period of time until a predetermined number of tests are administered, come up positive, come up positive across a predetermined geographic region, etc.

Also depicted in FIG. 1 is a medical supply monitoring service 110. In some examples, medical supply monitoring service 110 may be implemented on server(s) that form what is often referred to as a “cloud infrastructure” or “the cloud,” though this isn't true in all examples. Medical supply monitoring service 110 may be communicatively coupled with networked components of medical facilities 102_{1-N} via network(s) indicated generally at 120. Medical supply monitoring service 110 may implement a variety of components that perform selected aspects of the present disclosure. These components may be implemented using any combination of hardware and computer-readable instructions.

An inventory engine 114 may perform various actions associated with checking and/or replenishing medical supply inventories at medical facilities 102_{1-N}. In some examples, inventory engine 114 may receive messages from computing devices 104₁₋₂ and/or standalone medical testing devices 106_{N} that indicate a patient or subject has been determined to have an exogenous medical condition. Based on these messages, inventory engine 114 may itself perform, or may trigger performance of, a medical supply replenishment routine.

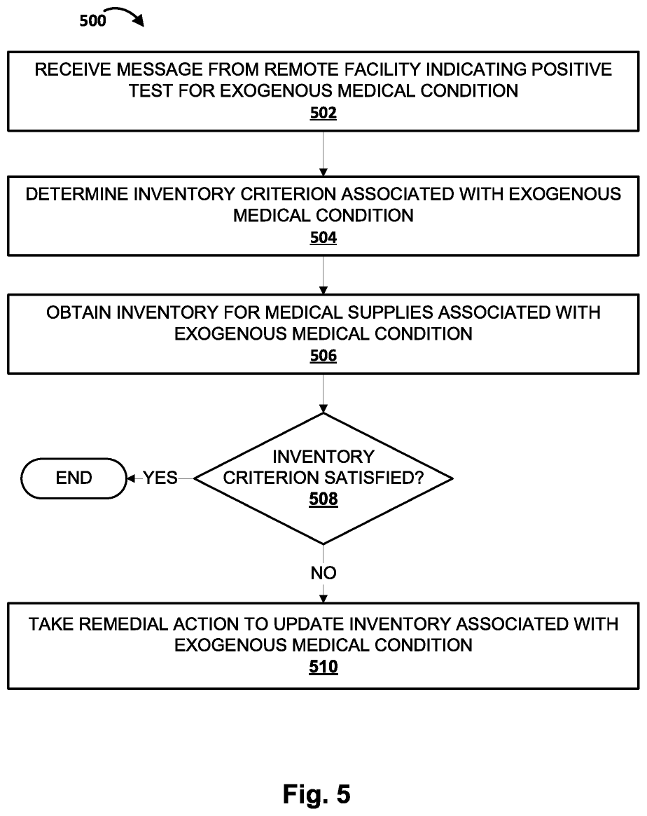

A routine to replenish medical supplies associated with the exogenous medical condition may seek to determine whether a given medical facility 102 has sufficient medical supplies associated with the exogenous medical condition. If the given medical facility 102 does not have adequate medical supplies, then various remedial actions may be taken. For example, medical supplies may be automatically ordered from a medical supplier, or may be automatically requested from another medical facility that has sufficient medical supplies. In some examples, multiple medical facilities may broadcast their inventories, e.g., to trigger an exchange of medical supplies between the medical facilities. In some examples, a notification may be sent/transmitted to personnel of the medical facility to prompt them to replenish the deficient medical supply. A non-limiting example of a routine to replenish medical supplies associated with the exogenous medical condition is depicted in FIG. 5.

Epidemiology engine 116 may determine when and/or whether messages should be transmitted to various medical facilities, e.g., based on the epidemiology of the exogenous medical condition under consideration. For example, epidemiology engine 116 may be more likely to transmit messages to various medical facilities upon detection of an extremely contagious and/or virulent pathogen. In some examples, epidemiology engine 116 may determine, e.g., from a local database (not depicted), a radius of potential impact of an exogenous medical condition. Based on that radius of potential impact, epidemiology engine 116 may determine whether and/or how far to transmit messages upon receiving a positive test result. In other examples, some of the operations may be performed locally at medical facilities, e.g., by computing devices 104.

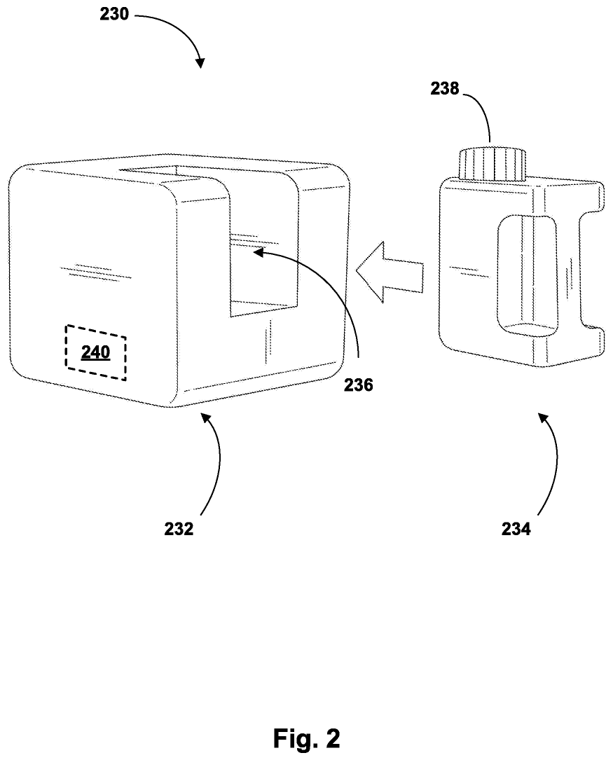

FIG. 2 depicts a non-limiting example of a medical testing device 230 configured with selected aspects of the present disclosure. Medical testing device 230 includes a sample compartment 234 (or “cartridge”) to receive a sample of a patient (not depicted). Medical testing device 230 also includes a base 232 (or “base component”) having a sensor (not depicted) to monitor interaction of the sample with a stimulus in sample compartment 234 and raise a signal indicative of a result of the interaction.

In some examples, sample compartment 234 may be removably installable in base 232 as indicated by the arrow in FIG. 2. For example, sample compartment 234 may be insertable into a slot 236 of base 232 that ensures the sensor is able to monitor the interaction occurring within sample compartment 234. Sample compartment 234 in FIG. 2 includes a cap 238 that is removable to insert the sample into an interior chamber of sample compartment 234. Medical testing device 230 is provided for illustrative purposes; other types of medical testing devices are contemplated.

Various types of patient samples may be used, such as tissue samples retrieve from biopsy, saliva, blood, urine, sweat, skin samples, hair samples, and so forth. The stimulus applied to the sample in sample compartment 234 may take various forms as well, depending on the type of sample, the exogenous medical condition being tested, the design of medical testing device 230, etc. In some examples, the stimulus is a chemical reagent that is exposed to the sample within sample compartment 234 to cause some sort of reaction, such as a color change, temperature change, etc. Other types of stimuli may be applied in other examples, including but not limited to electromagnetic radiation (e.g., various visible or invisible forms of light), electricity, temperature, etc.

Various types of sensors may be employed to detect the interaction between the stimulus and the sample. In some examples, the sensor may take the form of a light sensor that may or may not also include corresponding light emitter(s). In other examples, the sensor may take the form of, for instance, an electrical sensor, a thermometer, etc. The interaction, or a byproduct of the interaction, that is monitored by the sensor may be, for instance, a color change of the sample and/or reagent, a temperature change, a change in an electrical property such as a change in resistance, capacitance, etc. In some examples, sample compartment 234 may include a slide that can be used to hold the sample. The sensor may include a vision sensor that captures magnified image(s) of the slided sample and analyzes the images for presence of various biomarkers, microorganisms, etc.

In some examples, medical testing device 230 also includes logic 240. Logic 240 may take various forms, such as a microprocessor that executes computer-readable instructions stored in memory (not depicted in FIG. 2), an application-specific integrated circuit (“ASIC”), a field-programmable gate array (“FPGA”), etc. Logic 240 may be operably coupled with the sensor so that a signal raised by the sensor is received by logic 240.

In some examples, logic 240 may analyze the sensor signal to determine a result of a medical test administered using medical testing device 230. The result may indicate, for example, that the patient has an exogenous medical condition. In response, logic 240 may transmit a message to a computing device such as a computing device 104 in the same medical facility 102 as medical testing device 230 or a different medical facility. In various examples, the message may cause the computing device 104 to initiate a routine to replenish medical supplies associated with the exogenous medical condition at a medical facility.

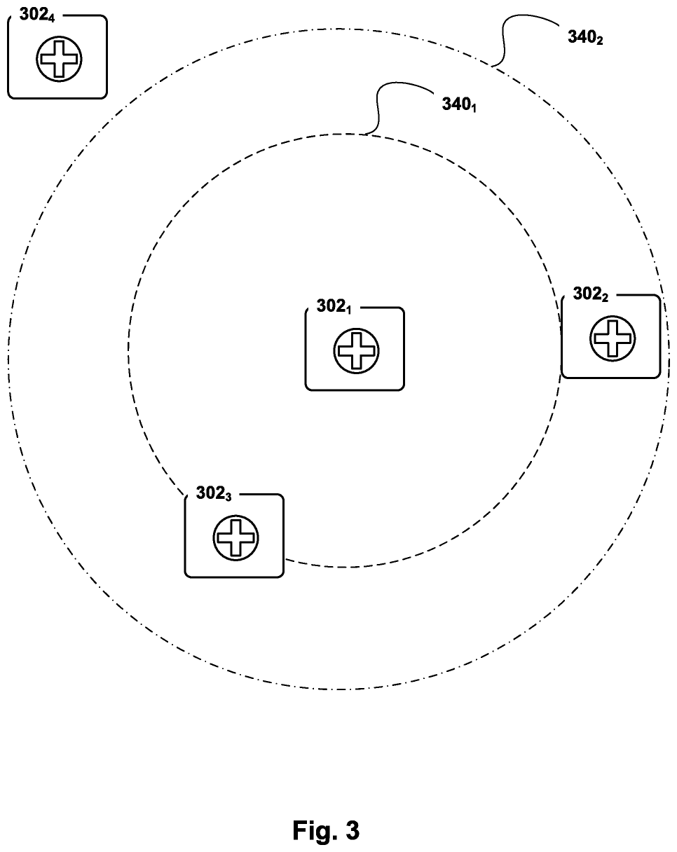

FIG. 3 schematically demonstrates how detected co-occurrence of an exogenous medical condition within a geographic area may trigger various aspects of the present disclosure. In FIG. 3, four medical facilities 302₁₋₄ are depicted across a geographic area for illustrative purposes, but this is not meant to be limiting. For purposes of demonstration, example scenarios will be described in which patients test positive for measles. However, this is not meant to be limiting, and techniques described herein may be applicable with any medical condition that is exogenous, i.e., caused by environmental factors. Thus, a “medical condition” as used herein may also include other contagious diseases, waterborne diseases, diseases caused by environmental phenomena such as pollution or radiation, etc.

Suppose the result of a measles test administered to a patient at a first medical facility 302₁ indicates the patient has measles. In some examples, this positive test result may cause a computing device or medical testing device at first medical facility 302₁ to transmit a message to other medical facilities, such as any of 302₂₋₄. This message may trigger a remote computing device at the recipient medical facility to initiate a routine to replenish medical supplies associated with measles.

In some examples, a computing device (e.g., 104) at first medical facility 302₁ and/or another computing device, such as medical supply monitoring service 110, may determine a radius 340₁ of potential impact of measles. A radius of potential impact of an exogenous medical condition may be determined in various ways. In some examples, each exogenous medical condition may be associated in a database (e.g., available to 110) with a radius of potential impact for that medical condition. Additionally or alternatively, in some examples, the radius of potential impact may be determined based on other signals, such as a number of positive tests run at a particular medical facility or amongst multiple medical facilities within a geographic area or region, a population density, weather conditions (which can affect how a disease is spread), etc.

In response to a positive result for the exogenous medical condition, the computing device (104 or 110) may select another medical facility (e.g., 302₃) based on the other medical facility being located within the radius 340₁ of potential impact of measles. For example, in FIG. 3, a third medical facility 302₃ is located at the radius 340₁ of potential impact of measles from first medical facility 302₁. Consequently, a computing device at first medical facility 302₁ may transmit a message to third medical facility 302₃ that triggers a computing device at third medical facility 302₃ to initiate a routine to replenish medical supplies associated with measles. Second and fourth medical facilities 302₂ and 302₄ are outside radius 340₁, and therefore are not sent messages.

Additionally or alternatively, a computing device such as medical supply monitoring service 110 may receive medical test results generated by a plurality of medical facilities. Based on the medical test results, computing device 110, e.g., by way of epidemiology engine 116, may identify co-occurrence of an exogenous medical condition within a geographic area. In response to identification of the co-occurrence, inventory engine 114 and/or epidemiology engine 116 may trigger replenishment of medical supplies associated with the exogenous medical condition at the medical facilit(ies).

In FIG. 3, for instance, suppose it is determined that positive tests for measles have been administered at first medical facility 302₁ and second medical facility 302₂. Because both these medical facilities lie within a second radius 340₂, the fact that measles is co-occurring between them may cause message(s) to be transmitted to other medical facilities within radius 340₂, such as third medical facility 302₃, to trigger a routine to replenish supplies associated with measles.

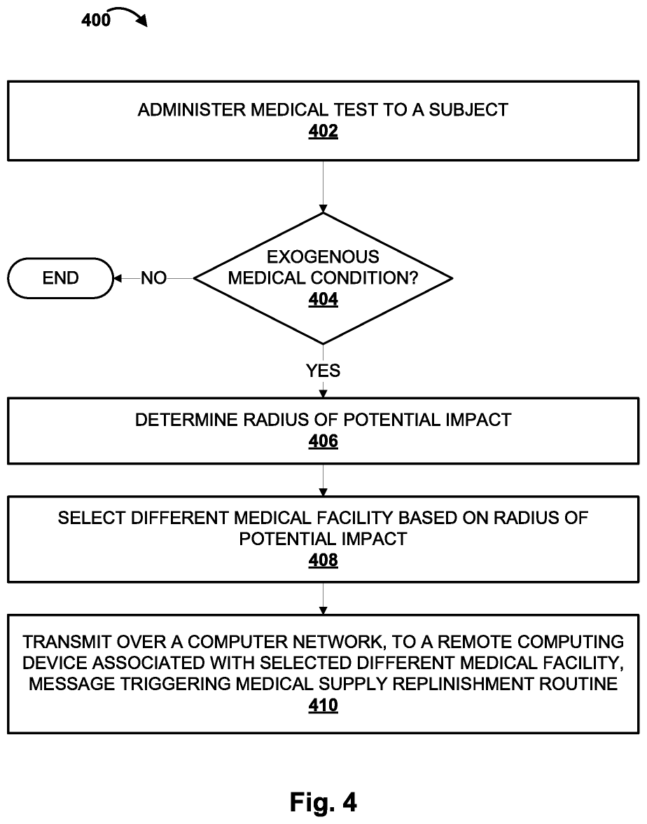

FIG. 4 depicts an example method 400 of practicing selected aspects of the present disclosure. For convenience, operations of method 400 will be described as being performed by a system configured with selected aspects of the present disclosure. The operations in FIG. 4 are not meant to be limiting; various operations may be added, omitted, and/or reordered.

At block 402, a medical test may be administered to a patient or subject to test for presence in the patient of an exogenous medical condition. For example, a medical testing device such as 230 in FIG. 2 may be operated by a clinician, nurse, etc., on a sample (e.g., skin, breath, blood, sweat, saliva, etc.) obtained from the patient. Medical testing device 230 may generate a result based on the administration of the medical test. The result may be audible or visual (or haptic) output, an outgoing network transmission, etc.

At block 404, the system may determine whether the test result generated at block 402 indicates that the patient has the exogenous medical condition. If the answer at block 404 is no, then method 400 may end. However, if the answer at block 404 is yes, then method may proceed to block 406.

At block 406, the system, e.g., by way of a computing device 104 at a medical facility 102 and/or epidemiology engine 116, may determine a radius of potential impact of the positively-tested exogenous medical condition. As noted above, in some examples this may include matching the exogenous medical condition to a record in a database that indicates how easily the medical condition is spread. In some examples, other signals may be taken into account when determining the radius of potential impact of the exogenous medical condition. For example, a current population density in the area may impact how easily and/or far the medical condition will likely spread. Also, factors such as whether the exogenous medical condition is spread through the air, water, human contact, food supply, etc., may be taken into account when the system determines the radius of potential impact of the exogenous medical condition. In some examples, signals such as the time-of-year, weather patterns, observed weather conditions, etc., may be considered when determining the radius of potential impact of an exogenous medical condition. For example, some contagious diseases spread more easily in the colder months because people spend more time indoors and therefore are more heavily exposed to others' pathogens.

In some examples, a statistical model and/or machine learning model such as a support vector machine, neural network, hidden Markov model, etc., may be trained to infer data indicative of the radius of potential impact of an exogenous medical condition. For example, the machine learning model may be applied to a plurality of inputs (e.g., an input vector) that includes various of the features mentioned above, such as how the disease is spread (air, water, physical contact, mucus, sexually), weather conditions (e.g., wind, temperature, precipitation), population density, population demographics, etc. The machine learning model may generate output that indicates, for instance, an inferred radius of potential impact of the exogenous medical condition.

In some such examples, the machine learning model may be trained using training data that is labeled with observed radii of potential impact of past outbreaks of the exogenous medical condition. A loss function of the machine learning model may be a difference, or “error,” between output generated from a training example's inputs and the label (e.g., observed radius of potential impact) applied to the training example. This loss function may be minimized by adjusting weights of the machine learning model using techniques such as gradient descent, back propagation, etc.

Referring back to FIG. 4, based on the radius of potential impact determined at block 406, at block 408, the system may select a different medical facility than the one at which the positive test result was detected. For example, in FIG. 3, if the positive test result is detected at first medical facility 302₁ and the radius of potential impact is 340₁, then third medical facility 302₃ may be selected, along with any other medical facilities within that radius of potential impact.

At block 410, the system may transmit, e.g., over computing network(s) (e.g., 120 in FIG. 1), to a remote computing device (e.g., 104) associated with the medical facility selected at block 408, a message. As noted above, this message may be anonymized so that the infected patient's identity is protected. Upon receipt at the remote computing device, the message may trigger a routine to replenish a medical supply at the selected other medical facility.

FIG. 5 depicts an example method 500 of practicing selected aspects of the present disclosure, namely, a routine for replenishing medical supplies associated with an exogenous medical condition. For convenience, operations of method 500 will be described as being performed by a system configured with selected aspects of the present disclosure, e.g., a computing system 104 at a medical facility (e.g., 102, 302). The operations in FIG. 5 are not meant to be limiting; various operations may be added, omitted, and/or reordered.

At block 502, the system may receive a message from a remote medical facility. The message may indicate that a patient at the remote medical facility tested positive for a particular exogenous medical condition. Notably, the message may be anonymized as described previously to ensure that the positive-testing patient's identity is not disclosed.

At block 504, the system may determine an inventory criterion associated with the exogenous medical condition. For a particularly contagious disease, the system may determine that a relatively large inventory of medical supplies for testing and/or treatment is needed when the contagious disease is detected elsewhere. On the other hand, a less-easily spread medical condition may not warrant as much inventory. If an exogenous medical condition is particular virulent, then a relatively large inventory of medical supplies for treating the exogenous medical condition may be warranted.

At block 506, the system may obtain an inventory for medical supplies associated with the exogenous medical condition at a particular medical facility. At block 508, the system may determine whether the inventory obtained at block 506 satisfies the inventory criterion determined at block 504. If the answer at block 508 is yes, then method 500 may end. However, if the answer at block 508 is no, then method 500 may proceed to block 510.

At block 510, the system may take remedial action to update an inventory associated with the exogenous medical condition in the medical facility. For example, an order for additional medical supplies may be placed automatically. Or, a notification may be provided, e.g., to medical facility personnel via text message, email, pop-up window, alarm, etc., that notifies the personnel that the inventory of medical supplies associated with the exogenous medical condition should be updated. As another example, other medical facilities may be queried for medical supplies, e.g., as part of a medical supply sharing routine.

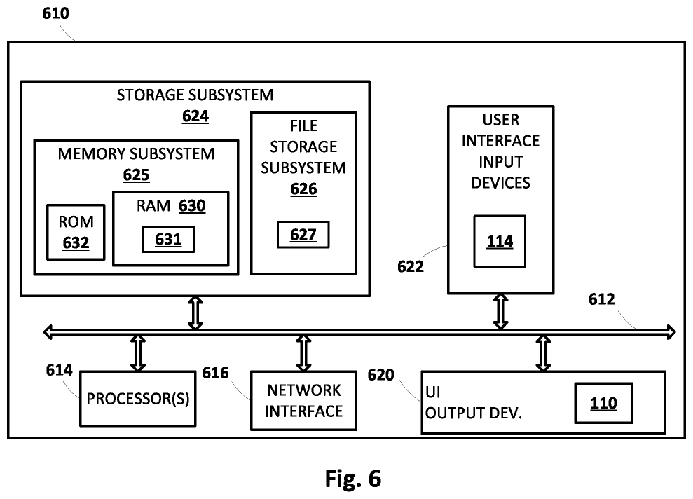

FIG. 6 is a block diagram of an example computer system 610. Computer system 610 may include a one processor 614 which communicates with a number of peripheral devices via bus subsystem 612. These peripheral devices may include a storage subsystem 624, including, for example, a memory subsystem 625 and a file storage subsystem 626, user interface output devices 620, user interface input devices 622, and a network interface subsystem 616. The input and output devices allow user interaction with computer system 610. Network interface subsystem 616 provides an interface to outside networks and is coupled to corresponding interface devices in other computer systems.

User interface input devices 622 may include input devices such as a keyboard, pointing devices such as a mouse, trackball, a touch interaction surface, a scanner, a touchscreen incorporated into the display, audio input devices such as voice recognition systems, microphone(s), vision sensor(s), and/or other types of input devices. In general, use of the term “input device” is intended to include all possible types of devices and ways to input information into computer system 610 or onto a communication network.

User interface output devices 620 may include a display subsystem, a printer, a fax machine, or non-visual displays such as audio output devices. The display subsystem may include a cathode ray tube (“CRT”), a flat-panel device such as a liquid crystal display (“LCD”), a projection device, or some other mechanism for creating a visible image. The display subsystem may also provide non-visual display such as via audio output devices. In general, use of the term “output device” is intended to include all possible types of devices and ways to output information from computer system 610 to the user or to another machine or computer system.

Storage subsystem 624 stores machine-readable instructions and data constructs that provide the functionality of some or all of the modules described herein. These machine-readable instruction modules are generally executed by processor 614 alone or in combination with other processors. Memory 625 used in the storage subsystem 624 may include a number of memories.

For example, a main random access memory (“RAM”) 630 may be used during program execution to store, among other things, instructions 631 for detecting and responding to positive medical tests for various exogenous medical conditions as described herein. Memory 625 used in the storage subsystem 624 may also include a read-only memory (“ROM”) 632 in which fixed instructions are stored.

A file storage subsystem 626 may provide persistent or non-volatile storage for program and data files, including instructions 627 for detecting and responding to positive medical tests for various exogenous medical conditions as described herein, and may include a hard disk drive, a floppy disk drive along with associated removable media, a CD-ROM drive, an optical drive, or removable media cartridges. The modules implementing the functionality of certain implementations may be stored by file storage subsystem 626 in the storage subsystem 626, or in other machines accessible by the processor(s) 614.

Bus subsystem 612 provides a mechanism for letting the various components and subsystems of computer system 610 communicate with each other as intended. Although bus subsystem 612 is shown schematically as a single bus, other implementations of the bus subsystem may use multiple busses.

Computer system 610 may be of varying types including a workstation, server, computing cluster, blade server, server farm, or any other data processing system or computing device. Due to the ever-changing nature of computers and networks, the description of computer system 610 depicted in FIG. 6 is intended as a specific example for purposes of illustrating some implementations. Many other configurations of computer system 610 are possible having more or fewer components than the computer system depicted in FIG. 6.

Although described specifically throughout the entirety of the instant disclosure, representative examples of the present disclosure have utility over a wide range of applications, and the above discussion is not intended and should not be construed to be limiting, but is offered as an illustrative discussion of aspects of the disclosure.

What is claimed is:

1. A method implemented by a computing device associated with a first medical facility, comprising:
determining, based on a result of a medical test administered to a subject, that the subject has an exogenous medical condition; and
in response to the determining, transmitting over a computer network, from the computing device associated with the first medical facility to a remote computing device associated with a second medical facility, a message that triggers the remote computing device to initiate a routine to replenish medical supplies associated with the exogenous medical condition at the second medical facility.

2. The method of claim 1, wherein the result is received at a communication interface of the computing device associated with the first medical facility from a medical testing device that administered the medical test.

3. The method of claim 2, wherein the result is received from a disposable component or a base component of the medical testing device.

4. The method of claim 1, wherein the message indicates detection of the medical condition of the subject, without identifying the subject.

5. The method of claim 1, wherein the result is received as input provided at a user interface of the computing device associated with the first medical facility.

6. The method of claim 1, wherein the exogenous medical condition comprises a contagious disease.

7. The method of claim 1, wherein the exogenous medical condition comprises a medical condition caused by an environmental phenomenon.

8. The method of claim 1, where in the medical supplies associated with the exogenous medical condition include fungible components of medical testing devices that administer the medical test.

9. The method of claim 1, further comprising;
determining a radius of potential impact of the exogenous medical condition; and
selecting the second medical facility based on the second medical facility being located within the radius of potential impact of the exogenous medical condition.

10. The method of claim 1, further comprising determining, based on a second result of a second medical test administered to a second subject in a third medical facility, that the second subject has the exogenous medical condition;
wherein the transmitting is further in response to determining that the second subject has the exogenous medical condition.

11. A medical testing device comprising:
a sample compartment to receive a sample of a patient;
a sensor to monitor interaction of the sample with a stimulus in the sample compartment and raise a signal indicative of a result of the interaction; and
logic to transmit, to a computing device in response to the result indicating the patient has an exogenous medical condition, a message that causes the computing device to initiate a routine to replenish medical supplies associated with the exogenous medical condition at a medical facility.

12. The medical testing device of claim 11, wherein the stimulus comprises one of a chemical reagent or electromagnetic radiation.

13. The medical testing device of claim 11, further comprising a base, wherein the sample compartment is removably installable in the base.

14. The medical testing device of claim 11, wherein the computing device is located at the medical facility.

15. A system comprising a processor and memory storing instructions that, in response to execution of the instructions by the processor, cause the processor to:
receive medical test results generated by a plurality of medical facilities;
based on the medical test results, identify co-occurrence of an exogenous medical condition within a geographic area; and
in response to identification of the co-occurrence, trigger replenishment of medical supplies associated with the exogenous medical condition at one of the plurality of medical facilities.
